(19) United States
(12) Patent Application Publication
(10) Pub. No.: US 2007/0270963 A1
(43) Pub. Date: Nov. 22, 2007

(54) Intervertebral implants and methods of use

(75) Inventors: Anthony J. Melkent, Memphis, TN (US); Lindsey Gardner Waugh, Memphis, TN (US); Jason A. Edie, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/412,330

(22) Filed: Apr. 27, 2006

(51) Int. Cl. A61F 2/44

(52) U.S. Cl. 623/17.11

(57) ABSTRACT

The present application discloses intervertebral implants and methods of using the implants. The implants may include inferior and superior surfaces, and a sidewall that together form the body of the implant. One or both of the inferior and superior surfaces may include tapered surfaces to facilitate distraction of adjacent vertebral members. The multiple tapered sections allows insertion via multiple surgical approaches using a single implant thereby negating the need to employ a plurality of approach-specific implants as is conventional.

### BACKGROUND

The present invention relates generally to vertebral implants, and more particularly to self-distracting intervertebral implants.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, and/or loss of motion.

Various procedures include replacing the entirety or a section of a vertebral member, the entirety or a section of an intervertebral disc, or both. One or more replacement implants may be inserted to replace the damaged vertebral members and/or discs. The implants may further include bone growth material to facilitate fusion of the implant to one or both adjacent vertebral members. The implant should provide for housing the bone growth material, and prevent inadvertent removal of the material from the implant.

### SUMMARY

The present application is directed to intervertebral implants with two or more tapered sections for insertion from multiple approach angles into the intervertebral space. The implant comprises a body with inferior and superior surfaces and a sidewall. A first tapered section is positioned at a first section of the body, and a second tapered section is positioned at a second section of the body. The first tapered section provides for insertion into the intervertebral space from a first approach angle. Likewise, the second tapered section provides for insertion from a second approach angle. The multiple tapered sections allows insertion via multiple surgical approaches using a single implant thereby negating the need to employ a plurality of approach-specific implants as is conventional.

### BRIEF DESCRIPTION OF THE DRAWINGS

FIG. 1 illustrates a side view of one embodiment of a vertebral implant inserted in a patient's spine.

FIG. 2 illustrates a perspective view of one embodiment of a vertebral implant.

FIG. 3 illustrates a lateral perspective view of the vertebral implant of FIG. 2.

FIG. 4 illustrates a lateral perspective view of another embodiment of a vertebral implant.

FIG. 5 illustrates a perspective view of one embodiment of a vertebral implant.

FIG. 6 illustrates a perspective view of one embodiment of a vertebral implant.

FIG. 7 illustrates a cross section view of an implant formed according to another embodiment.

FIG. 8 illustrates a perspective view of one embodiment of a vertebral implant according to one embodiment.

FIG. 9 illustrates a cross section view cut along line IX-IX of FIG. 8.

FIG. 10 illustrates a top view of an implant according to one embodiment.

### DETAILED DESCRIPTION

The present application relates to intervertebral implants. The implant includes outer walls that contact adjacent vertebral members. In one embodiment, the implants include a plurality of abutting tapered surfaces to distract the vertebral members during insertion. The tapered surfaces may facilitate insertion of the implant using an anterior, oblique, or lateral approach, or any combination of approaches. An inner chamber may be formed within the body to hold bone growth material. The implants may include one or more porous surfaces to allow the bone growth material inside of the implant to fuse with the vertebral members.

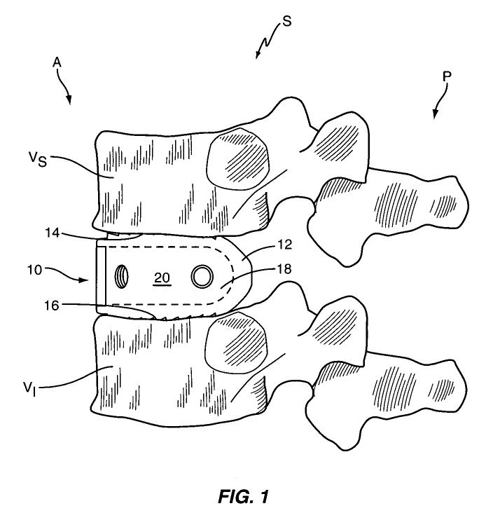

FIG. 1 illustrates a lateral view of one embodiment of an implant 10 positioned within a patient's spine S. For reference, this application indicates the anterior and posterior portions of a patient's body, and the implant 10, using the reference letters ‘A’ and ‘P,’ respectively. However, as described in more detail below, the implant 10 is not limited to any particular configuration or insertion approach with respect to the anterior and posterior portions of the patient.

As seen in FIG. 1, the implant 10 comprises a body 12 sized to fit within the intervertebral space between adjacent vertebral members V_{S}, V_{I}. In this embodiment, the body 12 is shaped to conform to the intervertebral space formed between the vertebral members V_{S}, V_{I}, however, other shapes are also possible. The body 12 includes superior surface 14, an inferior surface 16, and a surrounding sidewall 18. In one embodiment, the superior surface 14, the inferior surface 16, and the surrounding sidewall 18 substantially enclose an inner chamber 20. In FIG. 1, the superior surface 14 contacts the lower surface of the superior vertebral member V_{S}, while the inferior surface 16 contacts the upper surface the inferior vertebral member V_{I}. The inner chamber 20 may receive bone growth material that, as described below in more detail, grows through the superior and inferior surfaces 14, 16 to fuse with the vertebral members V_{S}, V_{I}.

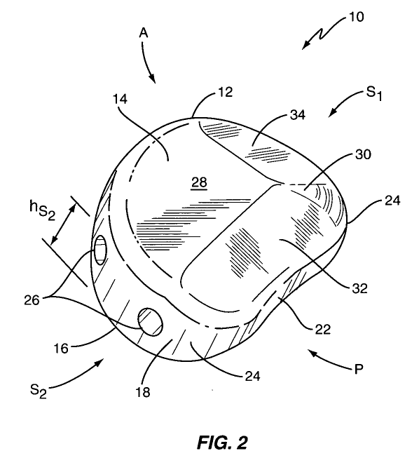

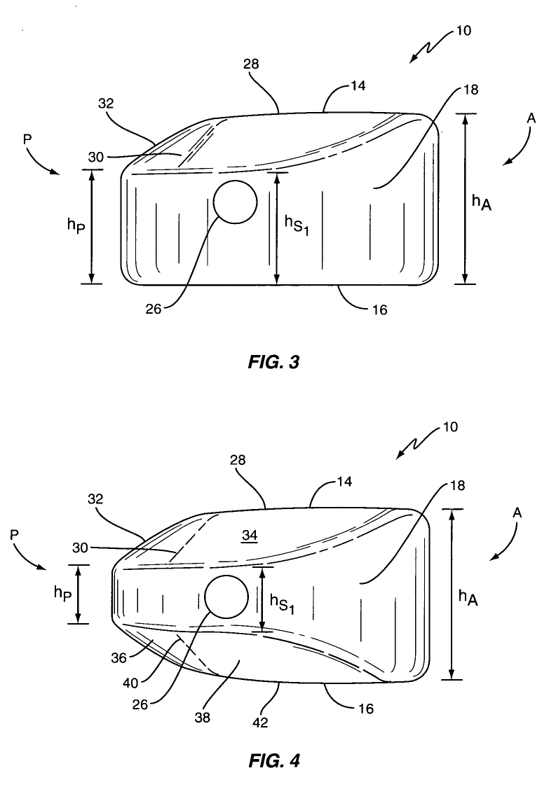

FIGS. 2 and 3 illustrate an implant 10 formed according to one embodiment. The implant 10 may be substantially kidney-shaped to match the shape of the vertebral members V_{S}, V_{I}, Other embodiments may feature the implant 10 including different shapes. As best seen in FIG. 2, the body 12 of implant 10 includes an anterior side A, a posterior side P, and two opposing lateral sides S₁, S₂. The sidewall 18 on the anterior side A and the opposing sides S₁, S₂ of body 12 may be generally arcuate to conform to the shape of the anterior and side portions of the vertebral members V_{S}, V_{I}. The sidewall 18 on the posterior side P of body 12 may include a concave section 22 and outer convex sections 24. One or more receptacles 26 may be formed on the sidewall 18 to receive any of a variety of insertion tools (not shown). The receptacles 26 may or may not be threaded, and may be formed at any desired location on the sidewall 18. Receptacles 26 may be positioned opposite from the tapered surfaces. In one embodiment, a first receptacle 26 is positioned on the sidewall 18 opposite from the first tapered surface 32, and a second receptacle 26 is positioned opposite from the second tapered surface 34.

In this embodiment, the inferior surface 16 of implant 10 is substantially flat (FIG. 3). Superior surface 14, however, includes a section 28 that is substantially flat and a third tapered surface 30 that extends between first and second tapered surfaces 32, 34. The substantially flat section 28 extends along a plane that is generally parallel to the inferior surface 16. The section 28 may include a greater surface area than either of the tapered surfaces 30, 32, 34. The first tapered surface 32 extends away from the section 28 to meet the peripheral edge of the sidewall 18 on the posterior side P of body 12. The second tapered surface 34 extends away from the section 28 to meet the peripheral edge of sidewall 18 on the lateral side S₁ of body 12. The third tapered surface 30 is positioned between the other surfaces and extends to meet the sidewall 18 between the posterior side P and lateral side S₁. As seen in FIGS. 2 and 3, the tapered surfaces 30, 32, 34 extend away from the first surface 28 in different directions. This reduces the heights h_{P}, h_{S1} of the body 12 on the posterior and lateral sides P, S₁, respectively, relative to the heights h_{A}, h_{S2} of the body 12 on the anterior and lateral sides A, S₂. Reducing the height on two different sides of the body 12 increases the functionality of the implant 10 by accommodating insertion from different directions. The tapered shape provides for the implant 10 to be self-distracting during insertion between the vertebral members V_{S}, V_{I}.

As best seen in FIG. 2, the first and second tapered surfaces 32, 34 slope downward from the section 28 at different angles. Particularly, the first tapered surface 32 slopes downward at a first angle, while the second tapered surface 34 slopes downward at a second, different angle. The third tapered surface 30 may include a varying angle to blend with the first and second tapered surfaces 32, 34. Constructing the implant 10 to include two tapered surfaces 32, 34, each sloping away from the section 28 at a different angle, reduces the heights h_{P} and h_{S1} by different amounts. By way of example, the first tapered surface 32 may slope at a more acute angle than the second tapered surface 34. Thus, the height h_{P} of sidewall 18 at the posterior side P of the body 12 would be smaller than the height h_{S1} of the sidewall at the lateral side S1 of body 12. These different heights for the sidewall 18 allows insertion via multiple surgical approaches using a single implant 10 thereby negating the need to employ a plurality of approach-specific implants as is conventional.

Those skilled in the art should note, however, that forming the tapered surfaces 30, 32, 34 to include different angles is not required. The tapered surfaces 30, 32, 34 may extend at any desired angle, and in some embodiments, the tapered surfaces 30, 32, 34 slope away from the section 28 at substantially the same angle. Even in embodiments in which the angles are the same, however, the tapered surfaces 30, 32, 24 allow the surgeon performing the insertion procedure the option of different surgical approaches without requiring a different implant.

The third tapered surface 30 comprises a smooth surface that extends between the section 28 and the peripheral edge of sidewall 18. The third tapered surface 30 extends along and between surfaces 32, 34, and thus, forms a substantially continuous surface between the first and second tapered surfaces 32, 34. In one embodiment, the third tapered surface 30 is slightly raised along each edge where it contacts the first and second tapered surfaces 32, 34 and extends downward from the section 28 to meet the peripheral edge of the sidewall 18. The angle at which the third tapered surface 30 extends is different from the angles at which the surfaces 32, 34 extend. In one embodiment, a centerline of the third tapered surface 30 extends substantially straight outward from the contact surface.

FIG. 4 illustrates an implant 10 according to another embodiment. In FIG. 4, the inferior surface 16 includes tapered surfaces 36, 38. Another tapered surface 40 extends away from a substantially flat portion 42 of inferior surface 16, and between the tapered surfaces 36, 38. As above, the angles at which the abutting tapered surfaces 36, 38, 40 extend away from the substantially flat portion 42 of the inferior surface 16 may be the same or different. The tapered surfaces 36, 38, 40 perform substantially the same function as the tapered surfaces 30, 32, 34 disposed on the superior surface 14. In addition, however, the tapered surfaces 36, 38, 40 on the inferior surface 16 further reduce the heights h_{P}, h_{S1} of the body 12 on the posterior and lateral sides P, S₁ relative to the heights h_{A}, h_{S2} of the body 12 on the anterior and lateral sides A, S₂.

In one embodiment, the body 12 includes a maximum height along a sidewall opposite from the tapered surfaces. The maximum height may also be located at other points along the body 12 including a central region or a point along the tapered surfaces.

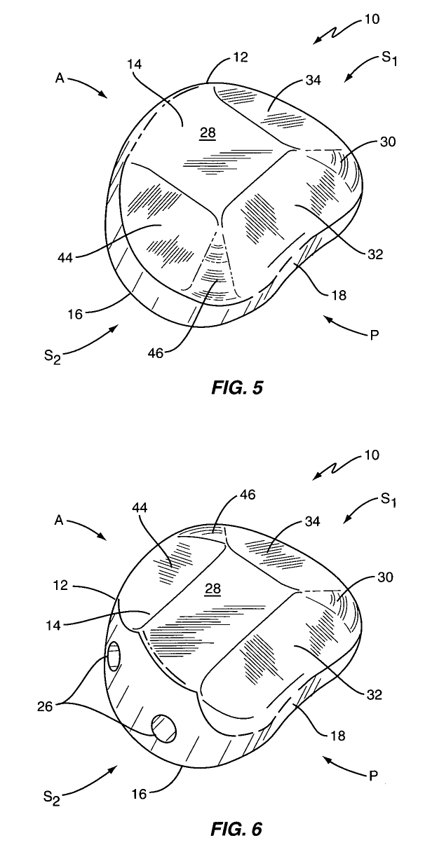

FIGS. 5 and 6 illustrate other embodiments of implant 10 wherein the superior surface 14 includes a fourth and fifth tapered surfaces 44, 46. In FIG. 5, the tapered surfaces 30, 32, 34, 44, 46 reduce the heights h_{P}, h_{S1}, and h_{S2} of the body 12 relative to the height h_{A} of the anterior side A of the body 12 to facilitate insertion from multiple approaches. Particularly, tapered surfaces 30, 32, 34 are disposed on the posterior side P and the lateral side S₁ of the body 12, respectively. Tapered surface 44 is disposed on the lateral side S₂ of body 12. As above, the angles at which each tapered surface 30, 32, 34, 44, 46 extend toward sidewall 18 may be the same or different.

FIG. 6 illustrates an embodiment wherein the tapered surfaces 30, 32, 34, 44, 46 reduce the heights h_{P}, h_{S1}, and h_{A} of the body 12 relative to the height h_{S2} of the lateral side S₂ of the body 12 to facilitate multiple insertion approaches. As seen in FIG. 6, tapered surfaces 44, 46 are disposed on the anterior side A of the body 12.

While not specifically shown in FIGS. 5 and 6, it should be noted that the inferior surface 16 may also be formed to include one or more tapered surfaces to compliment those tapered surfaces 30, 32, 34, 44, 46 disposed on the superior surface 14. In one embodiment, for example, the inferior surface 16 includes a corresponding number of tapered surfaces as those on the superior surface 14. In another embodiment, the inferior surface 16 may include different tapered surfaces.

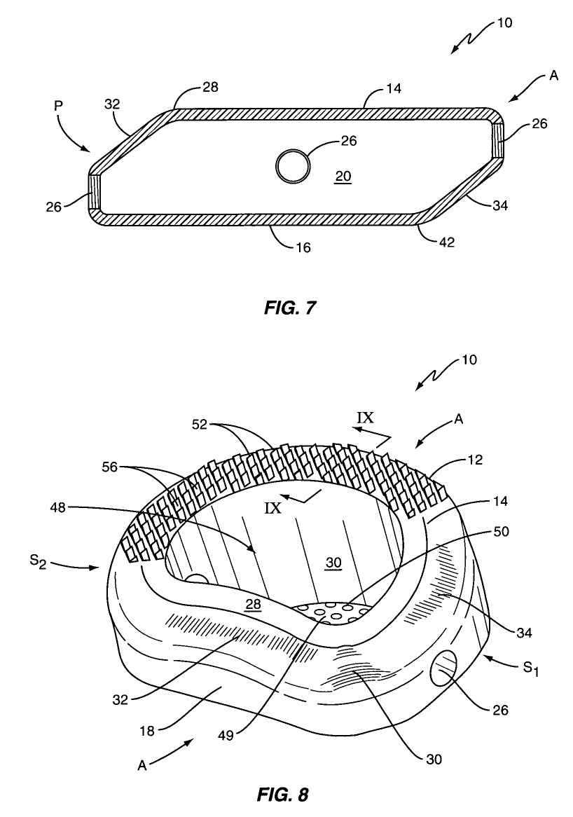

FIG. 7 illustrates another embodiment wherein the first tapered surface 32 is formed on the superior surface 14 at the posterior side P of the body 12, while the second tapered surface 34 is formed on the inferior surface 16 at the anterior side A of the body 12. As above, the angles at which the tapered surfaces 32, 34 extend from their respective sections 28, 42 may be the same or different. This embodiment facilitates the insertion of the implant 10 from either an anterior approach or a posterior approach. However, it should be noted that one or both of the superior and inferior surfaces 14, 16 may include additional tapered surfaces. These additional abutting tapered surfaces would, as described above, facilitate insertion and removal of the implant using multiple approaches. In one embodiment, the tapered surfaces on the inferior surface 16 overlap the tapered surfaces on the superior surface 14.

As previously stated, the implant 10 may be formed to promote fusion between the vertebral members V_{S}, V_{I}. In such embodiments, the implant 10 may be constructed to house bone growth material. In one embodiment (not illustrated), the body 12 includes an annular shape with an interior opening that extends through the body 12. FIG. 8 illustrates another embodiment with an opening 48 formed in the superior surface 14 that opens into an inner chamber 20. The inner chamber 20 is formed within the sidewalls 18 and a bottom wall 49. The inferior surface 16 may be formed by the outer side of the bottom wall 49. The bottom wall 49 may also include one or more openings 50 to promote fusion. A plurality of teeth 52 may be arranged on a portion of one or both of the superior and inferior surfaces 14, 16.

The opening 48 in the embodiment of FIG. 8 is enlarged to provide access to the inner chamber 20 so that the surgeon performing the surgical insertion procedure may pack the inner chamber 20 with bone growth material. In this embodiment, the inner chamber 20 is formed as a single cavity within an interior of the body 12. In other embodiments, however, the inner chamber 20 comprises a plurality of intercommunicating cavities that may be at least partially separated by one or more inner walls (not shown). In one embodiment, a cover (not illustrated) is sized to extend across the opening 48 and connect with the body 12.

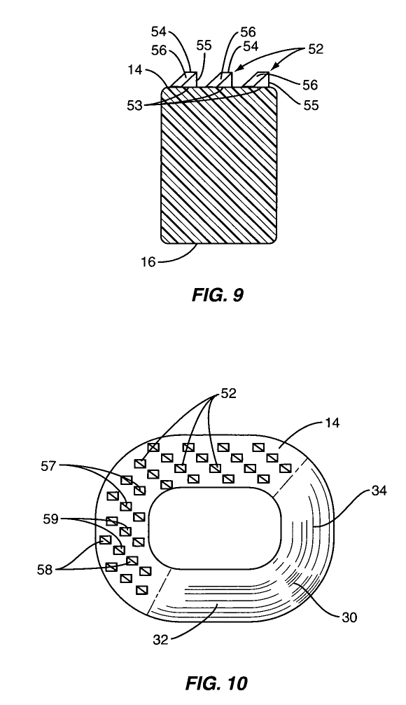

Teeth 52 may be distributed across one or both of the superior and inferior surfaces 14, 16. FIG. 9 illustrates the teeth 52 as they might be formed according to one embodiment. Each tooth 52 comprises a base 53 and an opposing tip 54. Each tooth 52 is further formed such that a length of a first side 55 is shorter than a length of a second side 56. Thus formed, the teeth 52 slant slightly towards one side of the body 12. This facilitates the insertion of the implant 10 into the intervertebral space. Once inserted, the tips 54 grip the surfaces of the vertebral members V_{S} and resist expulsion.

In one embodiment as illustrated in FIG. 8, the second side 56 of each tooth 52 is substantially aligned with the third tapered surface 30. This provides for the implant 10 to be inserted in a direction with either the first or second tapered surface 32, 34 being initially introduced into the intervertebral space.

FIG. 10 illustrates another embodiment of the teeth 52. One or more of the teeth 52 include a first angled surface 58 and a second angled surface 59. A ridge 57 extends between the angled surfaces 58, 59. The first angled surface 58 is aligned towards the first tapered surface 32, and the second angled surface 59 is aligned towards the second tapered surface 34. The first angled surface 58 facilitates insertion when the implant 10 is inserted with the first tapered surface 32. Likewise, the second angled surface 59 facilitates insertion with the second tapered surface 34. The angles surfaces 58, 59 may have the same or different shapes and sizes. In one embodiment, the ridge 57 of each of the teeth 52 is substantially aligned with the third tapered surface 30.

In the embodiments of FIGS. 8 and 10, each of the teeth 52 is substantially the same in shape and size. In other embodiments, a variety of different teeth are positioned on one or both of the surfaces 14, 16. The teeth 52 may be distributed across portions of one or both of the sections 28, 42 of superior and inferior surfaces 14, 16. In some embodiments, teeth 52 are also positioned along the tapered surfaces.

It should be noted that the figures illustrate the various receptacles 26 as being threaded. However, threaded receptacles 26 are not required. Each receptacle 26 described herein may be threaded or not threaded. Additionally, the body 12 may include both threaded and unthreaded receptacles 26.

In one embodiment, the sections 28, 42 are substantially flat. In other embodiments, one or both of the sections 28, 42 may be arcuate to match the contour of the vertebral members V_{S}, V_{I}.

Surgical approaches to the spine generally fall within broad categories of approaches. These are anterior, posterior, and lateral approaches, as well as combinations of these approaches such as anterior-lateral, posterior-lateral, and oblique approaches. Within each broad category of approach, there are numerous specific approaches tailored specifically for the cervical, thoracic, lumbar, sacral, and coccygela segment of the spine S to be surgically accessed. The preceding description has described the embodiments in the context of certain approaches. However, it should be understood that the implant 10 may be inserted between any two vertebral members V_{S}, V_{I} using any of these broad or specific approaches, or any combination of approaches.

A variety of materials may be positioned within the inner chamber 20 to facilitate fusion of the vertebral members. Suitable examples of bone growth promoting substances include bone morphogenic protein (BMP), LIM mineralization protein (LMP), demineralized bone matrix (DBM), mesenchymal stem cells, blood platelet gel, and biological materials. Other materials are disclosed in U.S. Patent Application Publication Nos. 2005/0203206 and 2006/0025861, each herein incorporated by reference.

The present invention may be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

What is claimed is:

1. A vertebral implant sized to fit within an intervertebral space between vertebral members, the implant comprising:
a body including superior and inferior surfaces and a sidewall;
a contact section positioned on one of the superior and inferior surfaces to contact one of the vertebral members when the implant is positioned within the intervertebral space;
a first tapered surface that extends in a first direction from the contact section to the sidewall;
a second tapered surface that extends in a second direction from the contact section to the sidewall, the second direction being different than the first direction; and
a third tapered surface positioned between and abutting the first and second tapered surfaces.

2. The implant of claim 1, wherein the contact section is substantially flat.

3. The implant of claim 1, wherein the contact section includes a greater surface area than either of the first, second, and third tapered surfaces.

4. The implant of claim 1, wherein the third tapered surface forms a smooth transition between the first and second tapered surfaces.

5. The implant of claim 1, wherein the first and second tapered surfaces extend outward from the contact section at different angles.

6. The implant of claim 1, further comprising a plurality of teeth positioned on the contact surface, each of the plurality of teeth including a first angled surface positioned towards the first tapered surface and a second angled surface positioned towards the second tapered surface.

7. The implant of claim 4, further comprising a plurality of teeth positioned on the contact surface, each of the plurality of teeth including an angled surface that is substantially aligned with the third tapered surface.

8. The implant of claim 1, further comprising a fourth tapered surface on one of the inferior and superior surfaces.

9. The implant of claim 1, wherein the sidewall further comprises a first receptacle opposite from the first tapered surface and a second receptacle opposite from the second tapered surface.

10. A vertebral implant sized to fit within an intervertebral space between vertebral members, the implant comprising:
a body including a contact surface and a surrounding sidewall;
a first tapered surface that extends from the contact surface in a first direction towards the sidewall;
a second tapered surface that extends from the contact surface in a second direction towards the sidewall; and
a third tapered surface extending along and between the first and second tapered surfaces, the third tapered surface being raised from the first and second tapered surfaces.

11. The implant of claim 10, wherein the contact surface is substantially flat.

12. The implant of claim 10, wherein a centerline of the third tapered extends substantially straight outward from the contact surface.

13. The implant of claim 10, further comprising an opening within an interior of the body to contain bone growth material.

14. The implant of claim 14, wherein the opening further comprises a bottom wall.

15. The implant of claim 10, wherein the sidewall further comprises a first receptacle opposite from the first tapered surface and a second receptacle opposite from the second tapered surface.

16. The implant of claim 10, further comprising teeth positioned on the contact surface, the teeth including a first angled surface aligned with the first tapered surface and a second angled surface aligned with the second tapered surface.

17. The implant of claim 10, further comprising a plurality of teeth positioned on the contact surface, each of the plurality of teeth including an angled surface that is substantially aligned with the third tapered surface.

18. A vertebral implant sized to fit within an intervertebral space between vertebral members, the implant comprising:
a body including a superior surface and an inferior surface;
a sidewall connecting the superior surface and the inferior surface;
a first set of three tapered surfaces extending between a first contact section of the superior surface and the sidewall; and
a second set of three tapered surfaces extending between a second contact section of the inferior surface and the sidewall.

19. The implant of claim 18, wherein the first set and the second set of tapered surfaces are positioned in an overlapping orientation.

20. The implant of claim 18, wherein the body further comprises a central opening to contain bone growth material.

21. A vertebral implant sized to fit within an intervertebral space between vertebral members, the implant comprising:
a body including superior and inferior surfaces and a sidewall;
a contact section positioned on one of the superior and inferior surfaces to contact one of the vertebral members when the implant is positioned within the intervertebral space;
a first tapered surface that extends in a first direction from the contact section to the sidewall;
a second tapered surface that extends in a second direction from the contact section to the sidewall, the second direction being different than the first direction;
a third tapered surface extending along and between the first and second tapered surfaces; and
a plurality of teeth positioned on the contact section and being aligned with the third tapered surface.

22. The implant of claim 21, wherein the plurality of teeth each include a first angled surface aligned with the first tapered surface and a second angled surface aligned with the second tapered surface.

23. The implant of claim 21, wherein each of the plurality of teeth includes an angled surface that is substantially aligned with the third tapered surface.

24. The implant of claim 21, further comprising a second plurality of teeth positioned on the contact section and including a shape that is different than the plurality of teeth.

25. The implant of claim 21, wherein the body further comprises a central opening to contain bone growth material.

26. The implant of claim 21, wherein the contact section is substantially flat.

27. The implant of claim 26, wherein the first tapered surface extends from the contact section at a first angle and the second tapered surface extends from the contact section at a second angle, the first and second angles being different.
